(19) United States
(12) Patent Application Publication
(10) Pub. No.: US 2019/0320931 A1
(43) Pub. Date: Oct. 24, 2019

(54) SIGNAL ACQUISITION DEVICE, SIGNAL ACQUISITION METHOD AND WEARABLE APPARATUS

(71) Applicants: CHONGQING BOE OPTOELECTRONICS TECHNOLOGY CO., LTD., Chongqing (CN); BOE TECHNOLOGY GROUP CO., LTD., Beijing (CN)

(72) Inventors: Ziwei WANG, Beijing (CN); Linglong LU, Beijing (CN); Yulong CHEN, Beijing (CN); Xiang ZHANG, Beijing (CN); Niannian WANG, Beijing (CN); Yong XIONG, Beijing (CN)

(21) Appl. No.: 16/241,588

(22) Filed: Jan. 7, 2019

(30) Foreign Application Priority Data
Apr. 23, 2018 (CN) 201810368287.1

(51) Int. Cl. A61B 5/0492; H03F 1/02; A61B 5/00; H03F 3/183

(52) U.S. Cl. CPC A61B 5/0492; H03F 3/183; A61B 5/6802; H03F 1/0205

(57) ABSTRACT

The present disclosure relates to a signal acquisition device, a signal acquisition method and a wearable apparatus. A preamplifier circuit in the signal acquisition device amplifies an obtained sEMG signal to produce an amplified sEMG signal. A baseline drift suppression circuit in the signal acquisition device extracts a first low-frequency component from the amplified sEMG signal and performs subtraction processing between the amplified sEMG signal and the first low-frequency component to obtain a difference signal. A filter circuit in the signal acquisition device filters the difference signal to obtain a target acquisition signal. With introduction of baseline drift suppression in the signal acquisition circuit, the baseline drift caused by a low-frequency component in a sEMG signal is eliminated, and the stability of a target acquisition signal is enhanced.

### CROSS REFERENCE TO RELATED APPLICATIONS

This U.S. non-provisional patent application claims priority under 35 U.S.C. § 119 to Chinese patent application No. 201810368287.1 filed on Apr. 23, 2018, the entire disclosure of which is incorporated herein by reference.

### FIELD

The present invention relates to the technical field of integrated circuit, and more particularly to a signal acquisition device, a signal acquisition method and a wearable apparatus.

### BACKGROUND

With the development of scientific technologies, there have emerged many high-tech electronic products, such as a wearable apparatus, in particular a wearable apparatus based on Surface Electromyography (sEMG) signals. Studies have always been conducted on sEMG signals for its application in the biomedical engineering field, such as prosthetic control, rehabilitation medicine and sports medicine, and clinical diagnosis. As biomedical technologies and artificial intelligence technologies develop, approaches for identifying gestures by using sEMG signals have been put forward and continuously explored. Studies on sEMG signal acquisition also become extremely important.

A sEMG signal is a weak signal on a human body surface. When a person wears a sEMG-signal-based wearable apparatus, target acquisition signals acquired by a signal acquisition device in the wearable apparatus are highly susceptible to interference from external signals. Muscular activities also tend to bring motion-induced interference noise. Meanwhile, substances, such as body liquid, generated on a human body surface will also interfere with the target acquisition signals. Those low-frequency interference signals are apt to lead to a baseline drift phenomenon, and affect the stability of the target acquisition signals.

### SUMMARY

According to a first aspect of the embodiments of the present disclosure, there is provided a signal acquisition device comprising a preamplifier circuit, a baseline drift suppression circuit and a filter circuit. The preamplifier circuit is configured to amplify an obtained sEMG signal to produce an amplified sEMG signal. The baseline drift suppression circuit comprises a low-frequency component extraction module and a subtraction module. The low-frequency component extraction module is configured to extract a first low-frequency component from the amplified sEMG signal. The subtraction module is configured to perform subtraction processing between the amplified sEMG signal and the first low-frequency component to obtain a difference signal. The filter circuit is configured to filter the difference signal to obtain a target acquisition signal.

Optionally, the low-frequency component extraction module comprises a first resistor, a second resistor, a first capacitor, a second capacitor and a first amplifier. A first terminal of the first resistor is connected with an output terminal of the preamplifier circuit to input the amplified sEMG signal, and a second terminal of the first resistor is connected with a first terminal of the second resistor. A second terminal of the second resistor is connected with a non-inverting input of the first amplifier. A first terminal of the first capacitor is connected with the second terminal of the first resistor, and a second terminal of the first capacitor is connected with an output terminal of the first amplifier. A first terminal of the second capacitor is connected with the second terminal of the second resistor, and a second terminal of the second capacitor is connected with ground. An inverting input of the first amplifier is connected with the output terminal of the first amplifier. The output terminal of the first amplifier outputs the first low-frequency component.

Optionally, the subtraction module comprises a third resistor, a fourth resistor, a fifth resistor, a sixth resistor and a second amplifier. A first terminal of the third resistor is connected with an output terminal of the low-frequency component extraction module to input the first low-frequency component, and a second terminal of the third resistor is connected with a non-inverting input of the second amplifier. A first terminal of the fourth resistor is connected with the second terminal of the third resistor, and a second terminal of the fourth resistor is connected with ground. A first terminal of the fifth resistor is connected with the output terminal of the preamplifier circuit to input the amplified sEMG signal, and a second terminal of the fifth resistor is connected with an inverting input of the second amplifier. A first terminal of the sixth resistor is connected with an output terminal of the second amplifier, and a second terminal of the sixth resistor is connected with the inverting input of the second amplifier. The output terminal of the second amplifier outputs the difference signal.

Optionally, the first low-frequency component has a frequency less than 20 HZ.

Optionally, the difference signal has a frequency greater than or equal to 20 HZ.

Optionally, the preamplifier circuit comprises a third amplifier and a seventh resistor. A non-inverting input of the third amplifier is connected with a first signal input terminal to input a first obtained sEMG signal, an inverting input of the third amplifier is connected with a second signal input terminal to input a second obtained sEMG signal, a reference signal terminal of the third amplifier is connected with a third signal input terminal to input a reference potential, and an output terminal of the third amplifier is connected with an input of the low-frequency component extraction module. A first power supply terminal of the third amplifier is connected with a first power supply, and a second power supply terminal of the third amplifier is connected with a second power supply. Two terminals of the seventh resistor are respectively connected to resistance terminals of the third amplifier and configured to control an amplification factor of the third amplifier. The output terminal of the third amplifier outputs the amplified sEMG signal.

Optionally, the filter circuit comprises a low-pass filter module and a power-frequency notch filter module. The low-pass filter module is configured to low-pass filter the difference signal to obtain a second low-frequency component of the difference signal. The power-frequency notch filter module is configured to perform power-frequency notch filtering on the second low-frequency component to obtain the target acquisition signal.

Optionally, the low-pass filter module comprises an eighth resistor, a ninth resistor, a tenth resistor, an eleventh resistor, a third capacitor, a fourth capacitor and a fourth amplifier. A first terminal of the eighth resistor is connected with an output terminal of the subtraction module to input the difference signal, and a second terminal of the eighth resistor is connected with a first terminal of the ninth resistor. A second terminal of the ninth resistor is connected with a non-inverting input of the fourth amplifier. A first terminal of the tenth resistor is connected with ground, and a second terminal of the tenth resistor is connected with an inverting input of the fourth amplifier. A first terminal of the eleventh resistor is connected with an output terminal of the fourth amplifier, and a second terminal of the eleventh resistor is connected with the inverting input of the fourth amplifier. A first terminal of the third capacitor is connected with the second terminal of the eighth resistor, and a second terminal of the third capacitor is connected with the output terminal of the fourth amplifier. A first terminal of the fourth capacitor is connected with the second terminal of the ninth resistor, and a second terminal of the fourth capacitor is connected with ground. A first power supply terminal of the fourth amplifier is connected with a first power supply, and a second power supply terminal of the fourth amplifier is connected with a second power supply. An output terminal of the fourth amplifier outputs the second low-frequency component of the difference signal.

Optionally, the power-frequency notch filter module comprises a twelfth resistor, a thirteenth resistor, a fourteenth resistor, a fifteenth resistor, a sixteenth resistor, a fifth capacitor, a sixth capacitor, a seventh capacitor, an eighth capacitor and a fifth amplifier.

A first terminal of the twelfth resistor is connected with an output terminal of the low-pass filter module, and a second terminal of the twelfth resistor is connected with a first terminal of the thirteenth resistor. The first terminal of the twelfth resistor is connected with the output terminal of the low-pass filter module to input the second low-frequency component of the difference signal, and the second terminal of the twelfth resistor is connected with a first terminal of the thirteenth resistor. A second terminal of the thirteenth resistor is connected with a non-inverting input of the fifth amplifier. A first terminal of the fifth capacitor is connected with the first terminal of the twelfth resistor, and a second terminal of the fifth capacitor is connected with a first terminal of the sixth capacitor. A second terminal of the sixth capacitor is connected with the second terminal of the thirteenth resistor. A first terminal of the seventh capacitor is connected with the second terminal of the twelfth resistor, and a second terminal of the seventh capacitor is connected with an output terminal of the fifth amplifier. A first terminal of the eighth capacitor is connected with the first terminal of the seventh capacitor, and a second terminal of the eighth capacitor is connected with the second terminal of the seventh capacitor. A first terminal of the fourteenth resistor is connected with the second terminal of the fifth capacitor, and a second terminal of the fourteenth resistor is connected with ground. A first terminal of the fifteenth resistor is connected with ground, and a second terminal of the fifteenth resistor is connected with the inverting input of the fifth amplifier. A first terminal of the sixteenth resistor is connected with the output terminal of the fifth amplifier, and a second terminal of the sixteenth resistor is connected with the second terminal of the fifteenth resistor. A first power supply terminal of the fifth amplifier is connected with a first power supply, and a second power supply terminal of the fifth amplifier is connected with a second power supply. The output terminal of the fifth amplifier outputs the target acquisition signal.

Optionally, the target acquisition signal has a frequency ranging from 20 HZ to 35 HZ, and from 65 HZ to 500 HZ.

Optionally, the signal acquisition device has a gain greater than 40 decibels and less than 65 decibels.

According to a second aspect of the embodiments of the present disclosure, there is provided a wearable apparatus. The wearable apparatus comprises an electromyography sensor for sensing a sEMG signal of a user who wears the wearable apparatus; a signal acquisition device as stated above for acquiring the sensed sEMG signal and generating a target acquisition signal based on the sEMG signal; a signal processing circuit for processing the target acquisition signal to identify the intention of the user; and an output circuit for providing a corresponding output based on the intention of the user.

Optionally, the outputting circuit comprises an Organic Light-Emitting Diode (OLED) display screen and/or an interface circuit.

According to a third aspect of the embodiments of the present disclosure, there is provided a signal acquisition method applicable to the signal acquisition device. The method comprises the steps of: amplifying an obtained sEMG signal to produce the amplified sEMG signal; extracting a first low-frequency component from the amplified sEMG signal; performing subtraction processing between the amplified sEMG signal and the first low-frequency component to obtain a difference signal; and filtering the difference signal to obtain a target acquisition signal.

### BRIEF DESCRIPTION OF DRAWINGS

FIG. 1 is a circuit structure schematic view of a signal acquisition device according to an embodiment of the present disclosure;

FIG. 2 is a circuit structure schematic view of a low-frequency component extraction module according to an embodiment of the present disclosure;

FIG. 3 is a circuit structure schematic view of a subtraction module according to an embodiment of the present disclosure;

FIG. 4 is a circuit structure schematic view of a preamplifier circuit according to an embodiment of the present disclosure;

FIG. 5 is a circuit structure schematic view of a low-pass filter module according to an embodiment of the present disclosure;

FIG. 6 is a circuit structure schematic view of a power-frequency notch filter module according to an embodiment of the present disclosure;

FIG. 7 is a structural schematic view of a signal acquisition device according to an embodiment of the present disclosure;

FIG. 8 is a schematic view of a target acquisition signal acquired by the signal acquisition device according to an embodiment of the present disclosure;

FIG. 9 is a structural schematic view of a wearable apparatus according to an embodiment of the present disclosure;

FIG. 10 is an actual product schematic view of the wearable apparatus according to an embodiment of the present disclosure; and

FIG. 11 is a flowchart showing a signal acquisition method according to an embodiment of the present disclosure.

### DETAILED DESCRIPTION

To make the objects, features and advantages of the present disclosure more obvious and understandable, the present disclosure will be described in detail with reference to the drawings and embodiments.

The present disclosure provides a signal acquisition device, a signal acquisition method and a wearable apparatus so as to solve the problem in the relevant art that the impact of a low-frequency interference signal on a target acquisition signal may easily lead to a baseline drift phenomenon.

The mechanism of a sEMG signal generation is as follows: voluntary contraction of normal skeletal muscle is caused by the excitement of cerebral cortex, and conducted by a nerve system. Since a nerve fibre and a muscle fibre are two completely different tissues and there is no direct cytoplasm (or protoplasm) therebetween, the transmission of nerve impulses is achieved by Acetylcholine (Ach). When a nerve ending senses a nerve impulse, Ach is released from synaptic vesicles for spreading, and enters into a synaptic groove to cause a muscle action potential, and soon cover the entire muscle fibre surface, which may even affect a locomotor system and thereby result to muscle contraction. Such an electrical change in a muscle fibre is called motor unit action potentials (MUAPs). The superposition of MUAPs in muscles in time and space form an Electromyography (EMG) signal. The comprehensive effect of an electrical activity on a nerve trunk and superficial muscle EMG generate a sEMG signal.

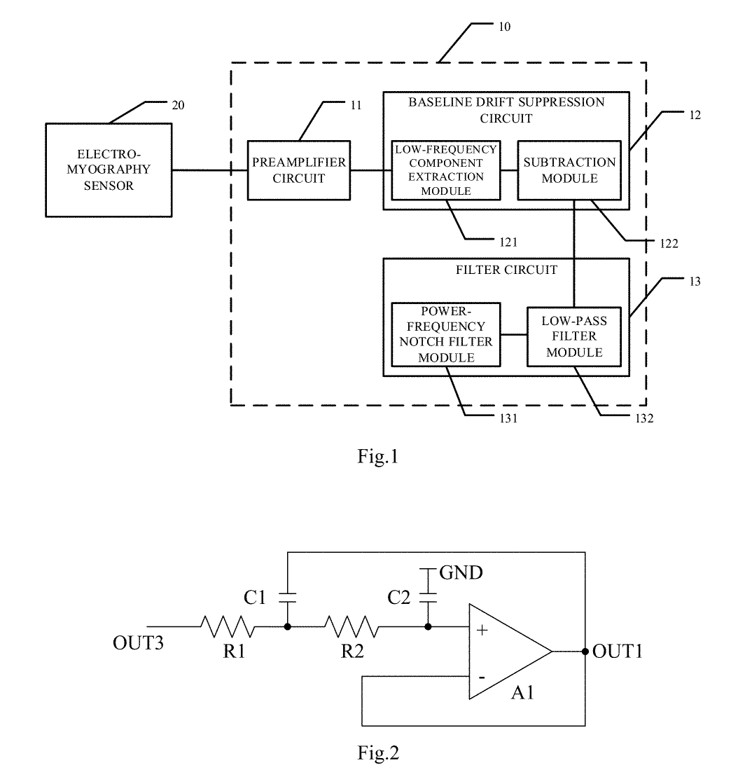

FIG. 1 illustrates a circuit structure schematic view of a signal acquisition device according to an embodiment of the present disclosure. As shown, the signal acquisition device 10 comprises a preamplifier circuit 11, a baseline drift suppression circuit 12 and a filter circuit 13.

The preamplifier circuit 11 is configured to amplify an obtained sEMG signal to produce an amplified sEMG signal. The baseline drift suppression circuit 12 comprises a low-frequency component extraction module 121 and a subtraction module 122. The low-frequency component extraction module 121 is configured to extract a first low-frequency component from the amplified sEMG signal. The subtraction module 122 is configured to perform subtraction processing between the amplified sEMG signal and the first low-frequency component to obtain a difference signal. The filter circuit 13 is configured to filter the difference signal to obtain a target acquisition signal.

With reference to FIG. 1, the preamplifier circuit 11 may be electrically connected with an electromyography sensor 20 to obtain a sEMG signal therefrom. The electromyography sensor 20 may be placed in a particular part of a human body, e.g. the part of muscle groups like finger extensor and finger flexor digitorum superficialis, so as to sense the sEMG signal on such muscle groups as finger extensor and finger flexor digitorum superficialis. Since a sEMG signal is a weak signal on a human body surface, the sEMG signal may be inputted to the preamplifier circuit 11 to be amplified by the preamplifier circuit 11, so as to produce the amplified sEMG signal.

It can be understood that the preamplifier circuit 11 may also be directly in electrical connection with any other suitable external signal source so as to obtain a sEMG signal therefrom. The external signal source may be, e.g., a signal generator or a silver halide dry electrode. Exemplarily, the preamplifier circuit 11 may be realized by an instrument amplifier.

A low-frequency interference signal may exist in the acquired sEMG signal due to an external low-frequency interference signal, a low-frequency interference signal caused by muscle activity and substances such as a body liquid generated by a human body surface, which may easily give rise to baseline drift of sEMG signals. The energy of those low-frequency interference signals will be greatly increased after being amplified by the preamplifier circuit 11, which will in turn affect the stability of a target acquisition signal. Hence, it is necessary to remove the low-frequency interference signal by the baseline drift suppression circuit 12, so as to eliminate baseline drift caused by low-frequency components.

In some embodiments, the low-frequency component extraction module 121 extracts the low-frequency interference signal from the amplified sEMG signal, i.e., to extract a first low-frequency component. Then, the subtraction module 122 perform subtraction processing between the amplified sEMG signal and the first low-frequency component to obtain a difference signal. As such, the baseline drift caused by the first low-frequency component is eliminated, and the stability of the target acquisition signal is improved. Finally, the filter circuit 13 filters the difference signal to filter out undesired signals from the difference signal and obtain the target acquisition signal.

In some embodiments, the low-frequency component extraction module 121 may be realized by a low-pass filter (such as Butterworth second-order low-pass filter).

It should be noted that in comparison with an approach for removing the first low-frequency component by utilizing a conventional filter, the approach for removing the first low-frequency component with the subtraction module 122 according to an embodiment of the present disclosure may remove the first low-frequency component more thoroughly. A conventional filter adopts an analog subtractor, and an analog circuit mainly operates on a time-domain voltage signal during a subtraction operation, so it is impossible to completely remove the first low-frequency component from the amplified sEMG signal. However, the subtraction module according to an embodiment of the present disclosure is inputted at its front end with a voltage signal in a specific frequency domain from the low-frequency component extraction module, which enables the subtraction module to perform voltage operation on the signal in the specific frequency domain. In doing so, the first low-frequency component can be completed removed from the amplified sEMG signal. Meanwhile, the subtraction module according to some embodiments of the present disclosure may utilize characteristics of an operational amplifier component (op-Amp), i.e., the input impedance approaching infinity and the output impedance being in the order of ohm, so as to effectively reduce an input current, thereby forming a micro-current environment, and achieving the purpose of greatly eliminating current (low-frequency component) interference.

FIG. 2 illustrates a circuit structure schematic view of a low-frequency component extraction module according to an embodiment of the present disclosure. As shown in FIG. 2, the low-frequency component extraction module 121 comprises a first resistor R1, a second resistor R2, a first capacitor C1, a second capacitor C2 and a first amplifier A1.

A first terminal of the first resistor R1 is connected with an output OUT3 of the preamplifier circuit 11. A second terminal of the first resistor R1 is connected with a first terminal of the second resistor R2. A second terminal of the second resistor R2 is connected with a non-inverting input of the first amplifier A1. A first terminal of the first capacitor C1 is connected with the second terminal of the first resistor R1. A second terminal of the first capacitor C1 is connected with an output OUT1 of the first amplifier A1. A first terminal of the second capacitor C2 is connected with the second terminal of the second resistor R2. A second terminal of the second capacitor C2 is connected with ground GND. An inverting input of the first amplifier A1 is connected with the output OUT1 of the first amplifier A1.

The low-frequency component extraction module as shown may receive the amplified sEMG signal from the output OUT3 of the preamplifier circuit 11, and output a low-pass filtered signal, that is, the first low-frequency component of the amplified sEMG signal, at the output OUT1 thereof.

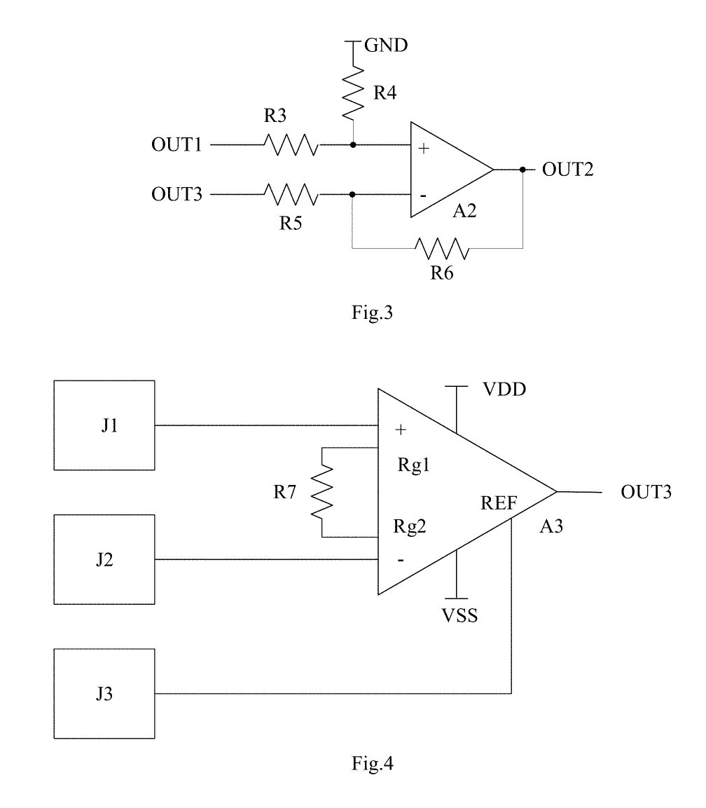

FIG. 3 illustrates a circuit structure schematic view of a subtraction module according to an embodiment of the present disclosure. As shown in FIG. 3, the subtraction module 122 comprises a third resistor R3, a fourth resistor R4, a fifth resistor R5, a sixth resistor R6 and a second amplifier A2.

A first terminal of the third resistor R3 is connected with an output OUT1 of the low-frequency component extraction module 121. A second terminal of the third resistor R3 is connected with a non-inverting input of the second amplifier A2. A first terminal of the fourth resistor R4 is connected with the second terminal of the third resistor R3. A second terminal of the fourth resistor R4 is connected with ground GND. A first terminal of the fifth resistor R5 is connected with the output OUT3 of the preamplifier circuit 11. A second terminal of the fifth resistor R5 is connected with an inverting input of the second amplifier A2. A first terminal of the sixth resistor R6 is connected with an output OUT2 of the second amplifier A2. A second terminal of the sixth resistor R6 is connected with the inverting input of the second amplifier A2.

The subtraction module as shown may receive the first low-frequency component of the amplified sEMG signal from the output OUT1 of the low-frequency component extraction module 121 and receive the amplified sEMG signal from the output OUT3 of the preamplifier circuit 11. Thus, the voltage at the output OUT2 of the second amplifier A2 is equal to a voltage obtained by subtracting the voltage at the output OUT1 of the low-frequency component extraction module 121 from the voltage at the output OUT3 of the preamplifier circuit 11.

In some embodiments, the resistance values of the first resistor R1 and the second resistor R2 and the capacitance values of the first capacitor C1 and the second capacitor C2 are so set that the frequency of the first low-frequency component extracted by the low-frequency component extraction module 121 is less than 20 HZ. The frequency of the difference signal obtained after the subtraction processing done by the subtraction module 122 may be made greater than or equal to 20 HZ by setting the resistance values of the third resistor R3, the fourth resistor R4, the fifth resistor R5 and the sixth resistor R6. It can be understood that the baseline drift suppression circuit 12 comprising the low-frequency component extraction module 121 and the subtraction module 122 produces no gain effect on the amplified sEMG signal.

The first amplifier A1 and the second amplifier A2 in embodiments of the present disclosure may be realized by, e.g., an AD8295 chip. The resistance value of the first resistor R1 may be 2.26KΩ, the resistance value of the second resistor R2 may be 2.26KΩ, the capacitance value of the first capacitor C1 may be 2.2 μF, the capacitance value of the second capacitor C2 may be 1 μF, and the resistance values of the third resistor R3, the fourth resistor R4, the fifth resistor R5 and the sixth resistor R6 may all be 20 KΩ.

The subtraction module subtracts a low-frequency component from the sEMG signal after the low-frequency component is separated from the sEMG signal by the low-frequency component extraction module, thereby counteracting the low-frequency energy. Since the low-frequency energy is counteracted, the low-frequency component extraction and the subtraction modules actually form a high-pass filter in combination, which in turn achieves the purpose of voltage filtering. Thus, the baseline drift suppression circuit according to some embodiments of the present disclosure not only improves the baseline drift phenomenon caused by current energy effectively, but also exerts the function of frequency domain voltage filtering.

FIG. 4 illustrates a circuit structure schematic view of a preamplifier circuit according to an embodiment of the present disclosure. As shown in FIG. 4, the preamplifier circuit 11 comprises a third amplifier A3 and a seventh resistor R7.

A non-inverting input of the third amplifier A3 is connected with a first signal input terminal J1. An inverting input of the third amplifier A3 is connected with a second signal input terminal J2. A reference signal terminal REF of the third amplifier A3 is connected with a third signal input terminal J3. An output OUT3 of the third amplifier A3 is connected with an input of the low-frequency component extraction module 121. A first power supply terminal of the third amplifier A3 is connected with a first power supply VDD. A second power supply terminal of the third amplifier A3 is connected with a second power supply VSS. Two terminals of the seventh resistor R7 are respectively connected to resistance terminals (Rg1 and Rg2 in FIG. 4) of the third amplifier A3. The seventh resistor R7 is configured to control an amplification factor of the third amplifier A3.

In some embodiments, when an electromyography sensor 20 is electrically connected with the preamplifier circuit 11, the electromyography sensor 20 comprises a first passive electrode, a second passive electrode and a third passive electrode. The first signal input terminal J1 may be the first passive electrode, the second signal input terminal J2 may be the second passive electrode, and the third signal input terminal J3 may be the third passive electrode. The sEMG signal at different parts can be acquired by the first passive electrode and the second passive electrode and then respectively inputted into the preamplifier circuit 11. A reference potential can be formed as a reference signal of the preamplifier circuit 11 by conductively connecting the third passive electrode with the human body. Since the entire preamplifier circuit is required to be connected to an analog ground (GND), the third passive electrode actually functions to provide an analog ground, and provides a reference potential for the entire circuit. Since the third passive electrode is located in proximity to two acquisition electrodes (namely, the first passive electrode and the second passive electrode), it may provide a relatively accurate regional reference potential. This further improves the accuracy of the sEMG signal.

Alternatively, when the preamplifier circuit 11 is in direct electrical connection with an external signal source, the external signal source may comprise a first signal source, a second signal source and a third signal source. The first signal source may input a sEMG signal to the first signal input terminal J1, the second signal source may input another sEMG signal to the second signal input terminal J2, and the third signal source may input a further sEMG signal to the third signal input terminal J3.

An amplification factor for the sEMG signal may be determined by setting a resistance value of the seventh resistor R7. Since the sEMG signal may comprise a signal at any frequency band and the preamplifier circuit 11 has no filtering function, the amplified sEMG signal also comprises a signal at any frequency band.

Exemplarily, when the resistance value of the seventh resistor R7 is 5.49 KΩ, a gain of the preamplifier circuit 11 is 20 decibels, namely, the amplified sEMG signal is obtained by amplifying the sEMG signal 20 times.

Returning to FIG. 1, as shown, in some embodiments, the filter circuit 13 comprises a low-pass filter module 131 and a power-frequency notch filter module 132. The low-pass filter module 131 is configured to low-pass filter the difference signal, i.e., to filter out the high-frequency component therein, to obtain a second low-frequency component of the difference signal. The power-frequency notch filter module 132 is configured to perform power-frequency notch filtering on the second low-frequency component to obtain the target acquisition signal.

In some embodiments, the low-pass filter module 131 may filter out undesired high-frequency components from the difference signal. The power-frequency notch filter module 132 may filter out the power-frequency interference from the difference signal to obtain a desired target acquisition signal.

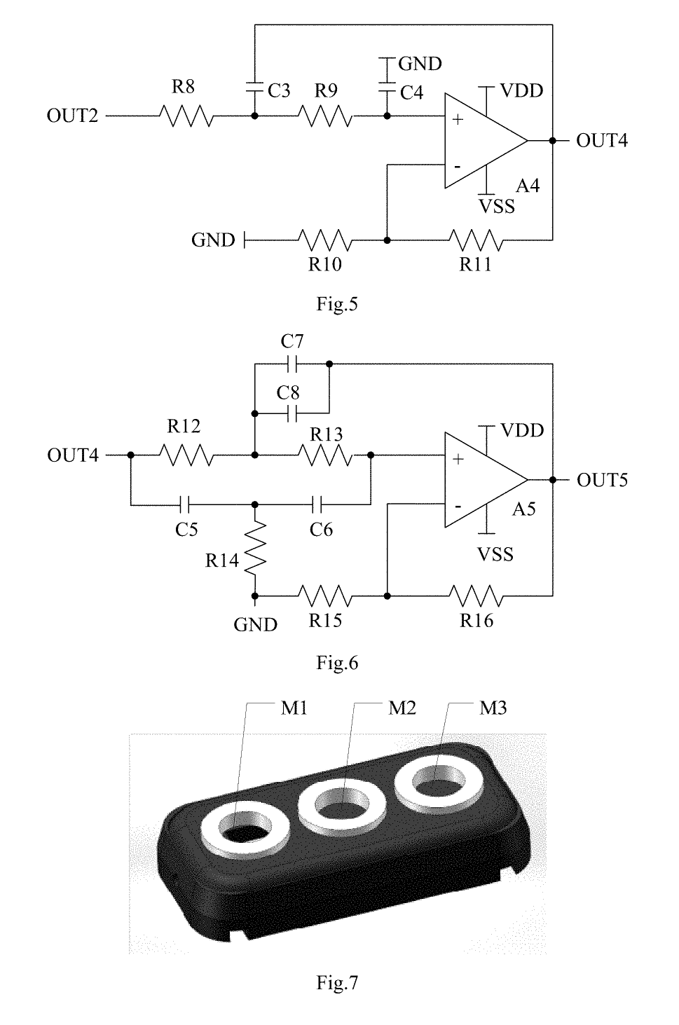

FIG. 5 illustrates a circuit structure schematic view of a low-pass filter module according to an embodiment of the present disclosure. As shown in FIG. 5, the low-pass filter module 131 comprises an eighth resistor R8, a ninth resistor R9, a tenth resistor R10, an eleventh resistor R11, a third capacitor C3, a fourth capacitor C4 and a fourth amplifier A4.

A first terminal of the eighth resistor R8 is connected with an output OUT2 of the subtraction module 122. A second terminal of the eighth resistor R8 is connected with a first terminal of the ninth resistor R9. A second terminal of the ninth resistor R9 is connected with a non-inverting input of the fourth amplifier A4. A first terminal of the tenth resistor R10 is connected with ground GND, and a second terminal of the tenth resistor R10 is connected with an inverting input of the fourth amplifier A4. A first terminal of the eleventh resistor R11 is connected with an output OUT4 of the fourth amplifier A4, and a second terminal of the eleventh resistor R11 is connected with the inverting input of the fourth amplifier A4. A first terminal of the third capacitor C3 is connected with the second terminal of the eighth resistor R8, and a second terminal of the third capacitor C3 is connected with the output OUT4 of the fourth amplifier A4. A first terminal of the fourth capacitor C4 is connected with the second terminal of the ninth resistor R9, and a second terminal of the fourth capacitor C4 is connected with ground GND. A first power supply terminal of the fourth amplifier A4 is connected with a first power supply VDD, and a second power supply terminal of the fourth amplifier A4 is connected with a second power supply VSS.

The difference signal can be low-pass filtered by setting the resistance values of the eighth resistor R8, the ninth resistor R9, the tenth resistor R10 and the eleventh resistor R11 and the capacitance values of the third capacitor C3 and the fourth capacitor C4, thereby mainly filtering out a signal component having a frequency greater than, e.g., 500 HZ. Thus, the obtained second low-frequency component of the difference signal has a frequency greater than or equal to 20 HZ, and less than or equal to 500 HZ.

Exemplarily, given that the resistance value of the eighth resistor R8 is 52.3 KΩ, the resistance value of the ninth resistor R9 is 7.32 Ω, the resistance value of the tenth resistor R10 is 2.49 KΩ, the resistance value of the eleventh resistor R11 is 97.6 KΩ, the capacitance value of the third capacitor C3 is 2.7 nF, and the capacitance value of the fourth capacitor C4 is 0.1 g, the low-pass filter module 131 has a low-pass cut-off frequency of 500 HZ, and has a gain of 32 decibels.

FIG. 6 illustrates a circuit structure schematic view of a power-frequency notch filter module according to an embodiment of the present disclosure. As shown in FIG. 6, the power-frequency notch filter module 132 comprises a twelfth resistor R12, a thirteenth resistor R13, a fourteenth resistor R14, a fifteenth resistor R15, a sixteenth resistor R16, a fifth capacitor C5, a sixth capacitor C6, a seventh capacitor C7, an eighth capacitor C8 and a fifth amplifier A5.

A first terminal of the twelfth resistor R12 is connected with an output OUT4 of the low-pass filter module 131, and a second terminal of the twelfth resistor R12 is connected with a first terminal of the thirteenth resistor R13. A second terminal of the thirteenth resistor R13 is connected with a non-inverting input of the fifth amplifier A5. A first terminal of the fifth capacitor C5 is connected with the first terminal of the twelfth resistor R12, and a second terminal of the fifth capacitor C5 is connected with a first terminal of the sixth capacitor C6. A second terminal of the sixth capacitor C6 is connected with the second terminal of the thirteenth resistor R13. A first terminal of the seventh capacitor C7 is connected with the second terminal of the twelfth resistor R12, and a second terminal of the seventh capacitor C7 is connected with an output OUTS of the fifth amplifier A5. A first terminal of the eighth capacitor C8 is connected with the first terminal of the seventh capacitor C7, and a second terminal of the eighth capacitor C8 is connected with the second terminal of the seventh capacitor C7. A first terminal of the fourteenth resistor R14 is connected with the second terminal of the fifth capacitor C5, and a second terminal of the fourteenth resistor R14 is connected with ground GND. A first terminal of the fifteenth resistor R15 is connected with ground GND, and a second terminal of the fifteenth resistor R15 is connected with the inverting input of the fifth amplifier A5. A first terminal of the sixteenth resistor R16 is connected with the output OUTS of the fifth amplifier A5, and a second terminal of the sixteenth resistor R16 is connected with the second terminal of the fifteenth resistor R15. A first power supply terminal of the fifth amplifier A5 is connected with a first power supply VDD, and a second power supply terminal of the fifth amplifier A5 is connected with a second power supply VSS.

The second low-frequency component may be power-frequency notch filtered by setting the resistance values of the twelfth resistor R12, the thirteenth resistor R13, the fourteenth resistor R14, the fifteenth resistor R15 and the sixteenth resistor R16, and the capacitance values of the fifth capacitor C5, the sixth capacitor C6, the seventh capacitor C7 and the eighth capacitor C8, thereby mainly filtering out a signal component having a frequency greater than 35 HZ and less than 65 HZ. The obtained target acquisition signal has a frequency ranging from 20 HZ to 35 HZ, and from 65 HZ to 500 HZ.

Exemplarily, given that the resistance value of the twelfth resistor R12 is 3.16 KΩ, the resistance value of the thirteenth resistor R13 is 3.16 KΩ, the resistance value of the fourteenth resistor R14 is 1.58 KΩ, the resistance value of the fifteenth resistor R15 is 2 KΩ, the resistance value of the sixteenth resistor R16 is 1.3 KΩ, and the capacitance values of the fifth capacitor C5, the sixth capacitor C6, the seventh capacitor C7 and the eighth capacitor C8 are all 1g, the power-frequency notch filter module 132 has a cut-off frequency greater than 35 HZ and less than 65 HZ, and has a gain of 3.74 decibels.

It should be noted that resistance values of resistors in the preamplifier circuit 11, the low-pass filter module 131 and the power-frequency notch filter module 132, and capacitance values of capacitors in the low-pass filter module 131 and the power-frequency notch filter module 132 are so adjusted that the gain of the signal acquisition device is greater than 40 decibels and less than 65 decibels.

In some embodiments, the voltage of the first power supply VDD is set to 5V, and the voltage of the second power supply VSS is set to −5V. The signal acquisition device 10 has, at 50 HZ, an input impedance of greater than 50 MΩ, and a common mode rejection ratio of greater than 60 decibels.

In some embodiments, the first terminal of the first resistor R1 in the low-frequency component extraction module 121 is connected with the output OUT3 of the third amplifier A3 in the preamplifier circuit 11. The first terminal of the third resistor R3 in the subtraction module 122 is connected with the output OUT1 of the first amplifier A1 in the low-frequency component extraction module 121. The first terminal of the fifth resistor R5 in the subtraction module 122 is connected with the output OUT3 of the third amplifier A3 in the preamplifier circuit 11. The first terminal of the eighth resistor R8 in the low-pass filter module 131 is connected with the output OUT2 of the second amplifier A2 in the subtraction module 122. The first terminal of the twelfth resistor R12 in the power-frequency notch filter module 132 is connected with the output OUT4 of the fourth amplifier A4 in the low-pass filter module 131.

Additionally, the first power supply VDD and the second power supply VSS are used in the third amplifier A3, the fourth amplifier A4 and the fifth amplifier A5. In order to guarantee the stability of the supplied DC voltage, it is also possible to arrange such components as a power supply filter circuit or parallel reference power supplies in the signal acquisition device, so as to process the DC voltage supplied from the first power supply VDD and the second power supply VSS, thereby improving the stability of the DC voltage. Meanwhile, in order to guarantee Electro Magnetic Compatibility (EMC) and Electromagnetic Interference (EMI) characteristics, the signal acquisition device 10 may further be isolated electromagnetically.

In some embodiments, the signal acquisition device 10 may be integrated on a Printed Circuit Board (PCB). Since the signal acquisition device 10 is highly integrated, the actual product size is relatively small. As such, the structure of the signal device unit 10 can be designed into an independent signal unit for inputting a target acquisition signal to a subsequent signal processing circuit.

FIG. 7 illustrates a schematic view showing an external configuration of a signal acquisition device according to an embodiment of the present disclosure. As shown in FIG. 7, the signal acquisition device may acquire multichannel parallel sEMG signals. Exemplarily, a first passive electrode, a second passive electrode and a third passive electrode may be respectively positioned at channels M1, M2 and M3, and are connected via a wire to an internal signal acquisition circuit integrated on the PCB.

Experimental results prove that a signal acquisition device in an embodiment of the present disclosure can truly and reliably obtain a target acquisition signal so as to provide a signal source to a back-end signal interface. In case that a wearable apparatus having a signal acquisition device according to an embodiment of the present disclosure is continuously worn, the signal acquisition device shows a good baseline property, and no baseline drift phenomenon occurs.

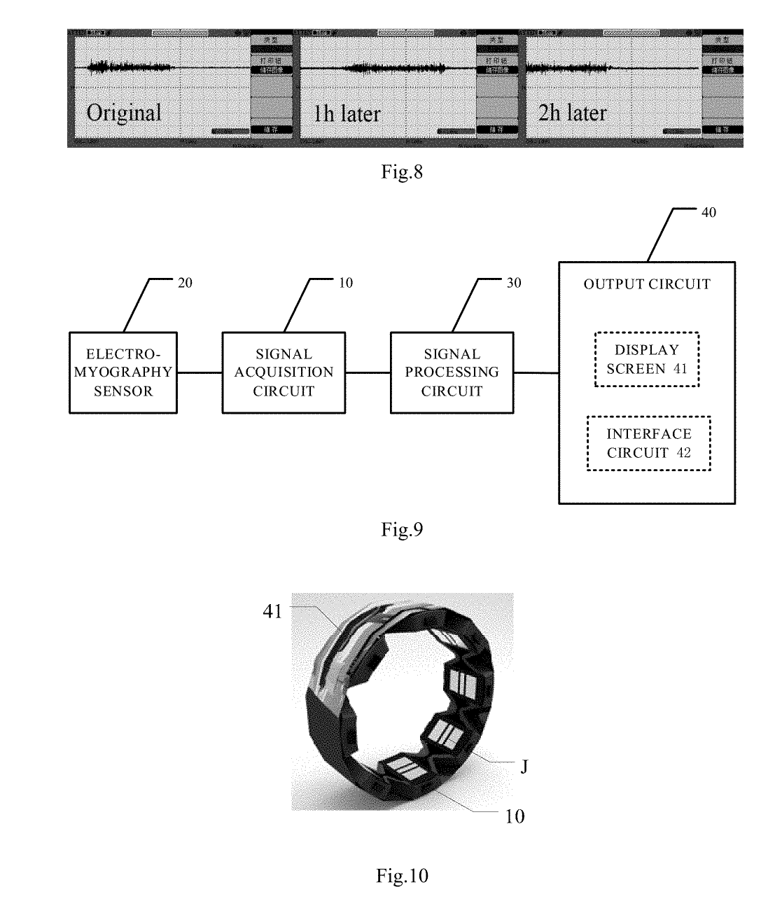

FIG. 8 illustrates a schematic view of a target acquisition signal acquired by a signal acquisition device according to an embodiment of the present disclosure. The left part, intermediate part and right part of FIG. 8 respectively shows the target acquisition signal acquired by the signal acquisition device 10 when the wearable apparatus is worn originally (Original in FIG. 8), one hour later (lh later in FIG. 8) and two hours later (2h later in FIG. 8). It can be seen that after the wearable apparatus has been worn continuously, the target acquisition signal acquired by the signal acquisition device 10 always has a good baseline property and no baseline drift occurs.

In embodiments of the present disclosure, a sEMG signal is amplified by a preamplifier circuit in a signal acquisition device to produce an amplified sEMG signal. A baseline drift suppression circuit in the signal acquisition device extracts a first low-frequency component from the amplified sEMG signal, and performs subtraction processing between the amplified sEMG signal and the first low-frequency component to obtain a difference signal. The difference signal is filtered by a filter circuit in the signal acquisition device to obtain a target acquisition signal. As such, the baseline drift is suppressed in the signal acquisition device, thereby eliminating the baseline drift caused by low-frequency components and improving the stability of the target acquisition signal.

FIG. 9 illustrates a structural schematic view of a wearable apparatus according to an embodiment of the present disclosure. As shown in FIG. 9, the wearable apparatus comprises a signal acquisition circuit 10, an electromyography sensor 20, a signal processing circuit 30 and an output circuit 40. The signal acquisition circuit 10 may be a signal acquisition device according to an embodiment of the present disclosure, such as a signal acquisition device as described above with reference to FIGS. 1 to 7.

The electromyography sensor 20 is for sensing a sEMG signal of a user who wears the wearable apparatus.

The signal acquisition circuit 10 is for obtaining the sensed sEMG signal from the electromyography sensor 20 and generating a target acquisition signal based on the sEMG signal.

The signal processing circuit 30 is for processing the target acquisition signal to identify an intention of the user. In some embodiments, the signal processing circuit 30 comprises an analog-to-digital conversion sub-circuit and a computation processing sub-circuit. The analog-to-digital conversion sub-circuit carries out the analog-to-digital conversion of the target acquisition signal generated by the signal acquisition circuit 10, so as to convert an analog target acquisition signal into a digital target acquisition signal. The digital target acquisition signal is transmitted to the computation processing sub-circuit (such as a classifier). The computation processing sub-circuit uses a pattern recognition algorithm, such as a Hidden Markov Model, to process the digital target acquisition signal in order to identify a real intention of the user and then transmit the real intention of the user to the signal output circuit 40.

The output circuit 40 is for providing a corresponding output based on the intention of the user. In some embodiments, the output circuit 40 comprises a display screen 41 (such as an OLED display screen) and/or an interface circuit 42. The interface circuit 42 may be a wireless interface circuit, such as Bluetooth, or WIFI (Wireless Fidelity).

The output signal as provided may be not only used for interaction with a user through the OLED display screen, but also used for signal interaction with other external peripheral via an interface circuit, for the purpose of entering into the IoT (Internet of Things) system.

In an exemplary scene, when a user wearing the wearable apparatus wants to check information on today's weather, he/she only needs to snap his/her fingers. The signal acquisition circuit 10 may output a target acquisition signal based on the acquired sEMG signal in relation to the finger-snapping action. The signal processing circuit 30 processes the target acquisition signal to analyze the user's true intention, namely, checking information on today's weather. Information on today's weather may then be displayed according to the user's intention through the OLED display screen.

In another exemplary scene, when the user wants to input contents into other device or system in the absence of a keyboard, he/she only needs to make a corresponding finger action in the air. Then, the wearable apparatus interacts with other apparatus or system by presenting, in the form of keyboard, results identified on the basis of the acquired sEMG signal in relation to the finger action, thereby turning the contents that the user wants to input into characters and entering them into other systems.

FIG. 10 illustrates an actual product schematic view of a wearable apparatus according to an embodiment of the present disclosure. The wearable apparatus is schematically shown as a wrist ring in a non-limiting way. The wrist ring comprises a plurality of passive electrodes J located at the parts of the wrist ring that are in contact with the wrist, a circuit part 10 within a body of the wrist ring and an OLED display screen 41 arranged on a surface of the wrist ring. The circuit part 10 may comprise a signal acquisition circuit, a signal processing circuit and an output circuit, etc., as described above.

A plurality of parallel sEMG signals are inputted by the plurality of passive electrodes J into the circuit part 10 in a multi-channel parallel manner, so as to import target acquisition signals from more channels. This may enhance the identification accuracy of target acquisition signals and reduce the time spent on identification. An identification result corresponding to an identified gesture may be shown on the OLED display screen 41.

In addition, the wearable apparatus in the embodiment of the present disclosure may also transmit, via an interface circuit, processing results of the signal processing circuit to a Mixed Reality (MR) apparatus, such as Microsoft Hololens. Since the current Microsoft Hololens device adopts gesture identification, it imposes limitation on the area of arm movement. When the wearable apparatus in the embodiment of the present disclosure is wirelessly connected with the Microsoft Hololens device, the processing results of the signal processing circuit may be transmitted to the Microsoft Hololens device, such that the Microsoft Hololens device is not subject to spatial restriction.

It can be understood that the wearable apparatus in the embodiment of the present disclosure may also be connected with an Apple Watch, a Virtual Reality (VR) device, an Augmented Reality (AR) device, etc.

In embodiments of the present disclosure, a baseline drift suppression circuit may be added in a signal acquisition circuit of a wearable apparatus, so as to eliminate the baseline drift caused by a first low-frequency component, thereby enhancing the stability of the target acquisition signal, and leading to more accurate identification and processing of a target acquisition signal by a signal processing circuit, which greatly improves the user experience.

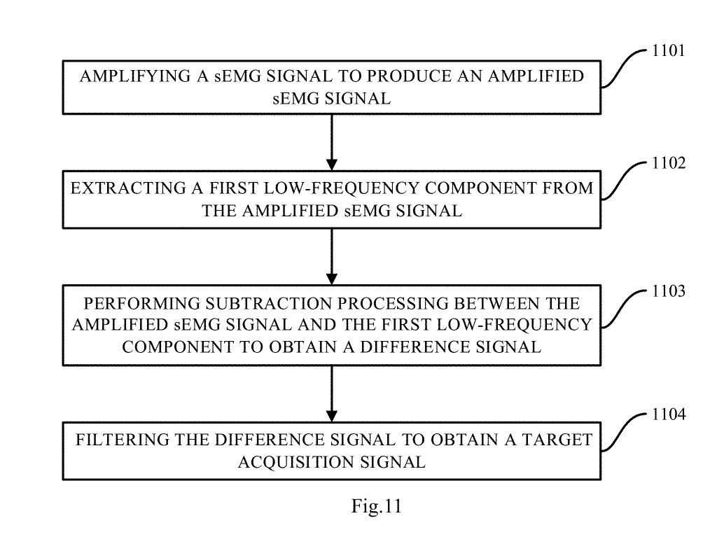

FIG. 11 illustrates a flowchart showing a signal acquisition method according to an embodiment of the present disclosure, which is applicable to the signal acquisition device according to an embodiment of the present disclosure.

Step 1101, an obtained sEMG signal is amplified to produce an amplified sEMG signal.

In some embodiments, the sEMG signal may be a sEMG signal of a human body sensed by an electromyography sensor placed in a particular part of the human body, e.g. muscle groups like finger extensor and finger flexor digitorum superficialis. Alternatively or additionally, the sEMG signal may come from an external signal source.

Optionally, the acquired sEMG signal may be amplified by a preamplifier circuit to produce an amplified sEMG signal.

Step 1102, a first low-frequency component is extracted from the amplified sEMG signal.

In the embodiment of the present disclosure, the first low-frequency component is to be eliminated as the first low-frequency component tends to cause baseline drift. The first low-frequency component in the amplified sEMG signal may be extracted by a low-frequency component extraction module.

Step 1103, subtraction processing between the amplified sEMG signal and the first low-frequency component is performed to obtain a difference signal. The difference signal is the one obtained by removing the first low-frequency component from the amplified sEMG signal.

In the embodiment of the present disclosure, after the first low-frequency component of the amplified sEMG signal is extracted by the low-frequency component extraction module 121, a subtraction module 122 performs subtraction processing between the amplified sEMG signal and the first low-frequency component to eliminate the first low-frequency component from the amplified sEMG signal so as to obtain the difference signal. This can eliminate the baseline drift caused by the low-frequency component and improve the stability of a target acquisition signal.

Step 1104, the difference signal is filtered to obtain a target acquisition signal.

In the embodiment of the present disclosure, a filter circuit 13 filters the difference signal so as to filter out undesired signals from the difference signal and obtain the target acquisition signal.

In the embodiment of the present disclosure, removal of undesired low-frequency components from the acquired sEMG signal can eliminate the baseline drift caused by the low-frequency component and improve the stability of the target acquisition signal.

To simplify the description, the above method embodiments are described as a series of combined actions. However, those skilled in the art shall know that the present disclosure is not limited by the sequence of actions. According to the present disclosure, some steps may be done in other sequence or simultaneously. Secondly, those skilled in the art shall also know that the embodiments described herein are alternative embodiments. The actions and modules involved are not necessarily required by the present disclosure.

The embodiments of the present description are described in a progressive manner, and each embodiment places emphasis on the difference it has with other embodiments, and reference can be made to other embodiments for identical or similar parts.

Finally, it should be noted that the relationship terms herein, such as first and second, are merely used to distinguish one entity or operation from another, and do not necessarily require or imply that there is any actual relationship or sequence between those entities or operations. In addition, the term “comprise”, “include” or any other variant thereof are non-exclusively inclusive, such that a process, method, product or apparatus including a series of elements includes not only those elements, but also other elements not explicitly listed, or further includes the elements intrinsic with such a process, method, product or apparatus. In the absence of further limitation, an element defined by the phrase “comprising a(an) . . . ” does not exclude that there are other identical elements in the process, method, product or apparatus comprising the element.

Circuits, modules and components in various embodiments may be implemented using hardware elements, software elements, or a combination of both. Examples of hardware elements may include devices, components, processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (such as a circuit or module), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application.

The embodiments of the present disclosure have been explained in detail. The description elaborates the principle and embodiments of the present disclosure by using specific examples. The above examples are explained to facilitate the understanding of the method and core concepts of the present disclosure. Meanwhile, as far as those ordinarily skilled in the art are concerned, changes can be made to specific embodiments and applicable scope according to the concepts of the present disclosure. In summary, the contents of the present description should not be understood as a limitation to the present disclosure.

1. A signal acquisition device comprising:
a preamplifier circuit configured to amplify an obtained surface electromyography (sEMG) signal to produce an amplified sEMG signal;
a baseline drift suppression circuit comprising a low-frequency component extraction module and a subtraction module, wherein the low-frequency component extraction module is configured to extract a first low-frequency component from the amplified sEMG signal, and wherein the subtraction module is configured to perform subtraction processing between the amplified sEMG signal and the first low-frequency component to obtain a difference signal; and
a filter circuit configured to filter the difference signal to obtain a target acquisition signal.

2. The signal acquisition device according to claim 1,
wherein the low-frequency component extraction module comprises a first resistor, a second resistor, a first capacitor, a second capacitor and a first amplifier,
wherein a first terminal of the first resistor is connected to an output terminal of the preamplifier circuit and is configured to receive the amplified sEMG signal, and a second terminal of the first resistor is connected to a first terminal of the second resistor,
wherein a second terminal of the second resistor is connected to a non-inverting input of the first amplifier,
wherein a first terminal of the first capacitor is connected to the second terminal of the first resistor, and a second terminal of the first capacitor is connected to an output terminal of the first amplifier,
wherein a first terminal of the second capacitor is connected to the second terminal of the second resistor, and a second terminal of the second capacitor is connected to ground, and
wherein an inverting input of the first amplifier is connected to the output terminal of the first amplifier, and
wherein the output terminal of the first amplifier is configured to output the first low-frequency component.

3. The signal acquisition device according to claim 1,
wherein the subtraction module comprises a third resistor, a fourth resistor, a fifth resistor, a sixth resistor and a second amplifier,
wherein a first terminal of the third resistor is connected to an output terminal of the low-frequency component extraction module and is configured to receive the first low-frequency component, and a second terminal of the third resistor is connected to a non-inverting input of the second amplifier,
wherein a first terminal of the fourth resistor is connected to the second terminal of the third resistor, and a second terminal of the fourth resistor is connected to ground,
wherein a first terminal of the fifth resistor is connected to the output terminal of the preamplifier circuit and is configured to receive the amplified sEMG signal, and a second terminal of the fifth resistor is connected to an inverting input of the second amplifier, and
wherein a first terminal of the sixth resistor is connected to an output terminal of the second amplifier, and a second terminal of the sixth resistor is connected to the inverting input of the second amplifier, and
wherein the output terminal of the second amplifier is configured to output the difference signal.

4. The signal acquisition device according to claim 1, wherein the first low-frequency component comprises a frequency less than 20 HZ.

5. The signal acquisition device according to claim 4, wherein the difference signal comprises a frequency greater than or equal to 20 HZ.

6. The signal acquisition device according to claim 1,
wherein the preamplifier circuit comprises a third amplifier and a seventh resistor,
wherein a non-inverting input of the third amplifier is connected to a first signal input terminal and is configured to recieve a first obtained sEMG signal,
wherein an inverting input of the third amplifier is connected to a second signal input terminal and is configured to receive a second obtained sEMG signal,
wherein a reference signal terminal of the third amplifier is connected to a third signal input terminal and is configured to receive a reference potential,
wherein an output terminal of the third amplifier is connected to an input of the low-frequency component extraction module,
wherein a first power supply terminal of the third amplifier is connected to a first power supply,
wherein a second power supply terminal of the third amplifier is connected to a second power supply,
wherein two terminals of the seventh resistor are respectively connected to resistance terminals of the third amplifier and configured to control an amplification factor of the third amplifier, and
wherein the output terminal of the third amplifier is configured to output the amplified sEMG signal.

7. The signal acquisition device according to claim 1, wherein the filter circuit comprises:
a low-pass filter module configured to low-pass filter the difference signal to obtain a second low-frequency component of the difference signal, and
a power-frequency notch filter module configured to perform power-frequency notch filtering on the second low-frequency component to obtain the target acquisition signal.

8. The signal acquisition device according to claim 7,
wherein the low-pass filter module comprises an eighth resistor, a ninth resistor, a tenth resistor, an eleventh resistor, a third capacitor, a fourth capacitor and a fourth amplifier,
wherein a first terminal of the eighth resistor is connected to an output terminal of the subtraction module and is configured to receive the difference signal, and a second terminal of the eighth resistor is connected to a first terminal of the ninth resistor,
wherein a second terminal of the ninth resistor is connected to a non-inverting input of the fourth amplifier,
wherein a first terminal of the tenth resistor is connected to ground, and a second terminal of the tenth resistor is connected to an inverting input of the fourth amplifier,
wherein a first terminal of the eleventh resistor is connected to an output terminal of the fourth amplifier, and a second terminal of the eleventh resistor is connected to the inverting input of the fourth amplifier,
wherein a first terminal of the third capacitor is connected to the second terminal of the eighth resistor, and a second terminal of the third capacitor is connected to the output terminal of the fourth amplifier,
wherein a first terminal of the fourth capacitor is connected to the second terminal of the ninth resistor, and a second terminal of the fourth capacitor is connected to ground, and
wherein a first power supply terminal of the fourth amplifier is connected to a first power supply,
wherein a second power supply terminal of the fourth amplifier is connected to a second power supply, and
wherein an output terminal of the fourth amplifier outputs the second low-frequency component of the difference signal.

9. The signal acquisition device according to claim 7,
wherein the power-frequency notch filter module comprises a twelfth resistor, a thirteenth resistor, a fourteenth resistor, a fifteenth resistor, a sixteenth resistor, a fifth capacitor, a sixth capacitor, a seventh capacitor, an eighth capacitor and a fifth amplifier,
wherein a first terminal of the twelfth resistor is connected to an output terminal of the low-pass filter module and is configured to receive the second low-frequency component of the difference signal, and a second terminal of the twelfth resistor is connected to a first terminal of the thirteenth resistor,
wherein a second terminal of the thirteenth resistor is connected to a non-inverting input of the fifth amplifier,
wherein a first terminal of the fifth capacitor is connected to the first terminal of the twelfth resistor, and a second terminal of the fifth capacitor is connected to a first terminal of the sixth capacitor,
wherein a second terminal of the sixth capacitor is connected to the second terminal of the thirteenth resistor,
wherein a first terminal of the seventh capacitor is connected to the second terminal of the twelfth resistor, and a second terminal of the seventh capacitor is connected to an output terminal of the fifth amplifier,
wherein a first terminal of the eighth capacitor is connected to the first terminal of the seventh capacitor, and a second terminal of the eighth capacitor is connected to the second terminal of the seventh capacitor,
wherein a first terminal of the fourteenth resistor is connected to the second terminal of the fifth capacitor, and a second terminal of the fourteenth resistor is connected to ground,
wherein a first terminal of the fifteenth resistor is connected to ground, and a second terminal of the fifteenth resistor is connected to the inverting input of the fifth amplifier,
wherein a first terminal of the sixteenth resistor is connected to the output terminal of the fifth amplifier, and a second terminal of the sixteenth resistor is connected to the second terminal of the fifteenth resistor,
wherein a first power supply terminal of the fifth amplifier is connected to a first power supply, and a second power supply terminal of the fifth amplifier is connected to a second power supply, and
wherein the output terminal of the fifth amplifier outputs the target acquisition signal.

10. The signal acquisition device according to claim 1, wherein the target acquisition signal has a frequency ranging from 20 HZ to 35 HZ, and/or from 65 HZ to 500 HZ.

11. The signal acquisition device according to claim 1, wherein the signal acquisition device has a gain greater than 40 decibels and less than 65 decibels.

12. A wearable apparatus, comprising:
an electromyography sensor configured to sense a surface electromyography (sEMG) signal of a user wearing the wearable apparatus;
a signal acquisition device according to claim 1 configured to obtain the sensed sEMG signal and configured to generate a target acquisition signal based on the sEMG signal that was sensed,
a signal processing circuit configured to process the target acquisition signal to identify an intention of the user, and
an output circuit configured to provide a corresponding output based on the intention of the user.

13. The wearable apparatus according to claim 12, wherein the output circuit comprises an Organic Light-Emitting Diode (OLED) display screen and/or an interface circuit.

14. The wearable apparatus according to claim 12,
wherein the low-frequency component extraction module comprises a first resistor, a second resistor, a first capacitor, a second capacitor and a first amplifier,
wherein a first terminal of the first resistor is connected to an output terminal of the preamplifier circuit and is configured to receive the amplified sEMG signal, and a second terminal of the first resistor is connected to a first terminal of the second resistor,
wherein a second terminal of the second resistor is connected to a non-inverting input of the first amplifier,
wherein a first terminal of the first capacitor is connected to the second terminal of the first resistor, and a second terminal of the first capacitor is connected to an output terminal of the first amplifier,
wherein a first terminal of the second capacitor is connected to the second terminal of the second resistor, and a second terminal of the second capacitor is connected to ground,
wherein an inverting input of the first amplifier is connected to the output terminal of the first amplifier, and
wherein the output terminal of the first amplifier outputs the first low-frequency component.

15. The wearable apparatus according to claim 12,
wherein the subtraction module comprises a third resistor, a fourth resistor, a fifth resistor, a sixth resistor and a second amplifier,
wherein a first terminal of the third resistor is connected to an output terminal of the low-frequency component extraction module and is configured to receive the first low-frequency component, and a second terminal of the third resistor is connected to a non-inverting input of the second amplifier,
wherein a first terminal of the fourth resistor is connected to the second terminal of the third resistor, and a second terminal of the fourth resistor is connected to ground,
wherein a first terminal of the fifth resistor is connected to the output terminal of the preamplifier circuit and is configured to receive the amplified sEMG signal, and a second terminal of the fifth resistor is connected to an inverting input of the second amplifier,
wherein a first terminal of the sixth resistor is connected to an output terminal of the second amplifier, and a second terminal of the sixth resistor is connected to the inverting input of the second amplifier, and
wherein the output terminal of the second amplifier outputs the difference signal.

16. The wearable apparatus according to claim 12, wherein the filter circuit comprises:
a low-pass filter module configured to low-pass filter the difference signal to obtain a second low-frequency component of the difference signal, and
a power-frequency notch filter module configured to perform power-frequency notch filtering on the second low-frequency component to obtain the target acquisition signal.

17. A signal acquisition method, performed by the signal acquisition device according to claim 1, the signal acquisition method comprising:
amplifying an obtained surface electromyography (sEMG) signal to produce the amplified sEMG signal,
extracting a first low-frequency component from the amplified sEMG signal,
performing subtraction processing between the amplified sEMG signal and the first low-frequency component to obtain a difference signal, and
filtering the difference signal to obtain a target acquisition signal.

18. The signal acquisition method according to claim 17, wherein the first low-frequency component comprises a frequency less than 20 HZ.

19. The signal acquisition method according to claim 17, wherein the difference signal comprises a frequency greater than or equal to 20 HZ.

20. The signal acquisition method according to claim 17, wherein said filtering the difference signal comprises:
low-pass filtering the difference signal to obtain a second low-frequency component of the difference signal, and
performing power-frequency notch filtering on the second low-frequency component to obtain the target acquisition signal.
